(19) United States
(12) Patent Application Publication
(10) Pub. No.: US 2026/0203335 A1
(43) Pub. Date: Jul. 16, 2026

(54) SYSTEMS AND METHODS FOR CLINICAL GRAPH DATA GENERATION USING ARTIFICIAL INTELLIGENCE MODELS

(71) Applicant: Guide Genetics, Inc., Niskayuna, NY (US)

(72) Inventors: Ming Kang, Seattle, WA (US); Matthew Robert Liebers, Philadelphia, PA (US); Saleem Ameen, Narwee (AU); Vladimir Bok, Burnaby (CA)

(21) Appl. No.: 19/444,962

(22) Filed: Jan. 9, 2026

Related U.S. Application Data
(60) Provisional application No. 63/743,636, filed on Jan. 10, 2025.

(51) Int. Cl. G06F 16/34; G06F 16/3329; G06F 40/169; G10L 13/08

(52) U.S. Cl. CPC G06F 16/345; G06F 16/3329; G06F 40/169; G10L 13/08

(57) ABSTRACT

Systems and methods for generating and presenting clinical data are disclosed. In some examples, an artificial intelligence agent generates domain-specific annotated data. A first dual prompt is then generated that includes a first prompt to identify identities, relationships of the identities, and clinical features of the identities, and a second prompt to generate a data object based on the annotated data. The first dual prompt is inputted to a large language model (LLM) to generate the data object. A second dual prompt is generated that includes a third prompt to generate a normalized clinical schema, and a fourth prompt to correct inconsistencies of the data object. The second dual prompt is inputted to the LLM to generate the normalized clinical schema, and graph node data is generated based on the normalized clinical schema and in a structured format. A dashboard then displays information based on the graph node data.

### CROSS-REFERENCE TO RELATED APPLICATION

This application claims priority to Application No. 63/743,636, filed on Jan. 10, 2025, and entitled “Method and Apparatus for AI-Driven Family History Taking, Real-Time Voice Transcription Pedigree Generation, and User Interface for Interactive Editing of Pedigree Diagrams,” the entire contents of which are incorporated herein by reference.

### Technical Field

This application relates generally to automated processes for the collection of clinical data, such as the collection of family history data for genetic evaluation.

### BACKGROUND

Traditionally, in an effort to collect clinical data, medical professionals interview patients. In at least some cases medical professionals have patients fill out questionnaire forms to gather the information. In some examples, the patients enter in the information to a device such as a tablet. These methods, however, are time consuming and can lead to errors. For instance, the patient may enter in the information incorrectly, or the medical professional may incorrectly record the patient's response. Moreover, the amount of information needing to be collected and evaluated for various clinical reasons is on the rise, such as the rise in the collection of clinical data for genetic testing. As such, there are opportunities to address technological deficiencies associated with the collection of clinical data and the summarization and evaluation of that clinical data.

### SUMMARY

Systems and methods for generating clinical graph data are disclosed. In some examples, an artificial intelligence (AI) agent generates domain-specific annotated data. For instance, the AI agent can use text-to-speech technology to convert text data characterizing questions into audio data, and the audio data is then transmitted to a user's device during a call. The user can respond to the questions, and the user's device transmits the response audio data during the call to the AI agent. The AI agent may then use speech-to-text technology to transcribe the user's responses to text data, and input at least portions of the text data to a large language model (LLM) to generate follow-up questions for the user for the specific domain (e.g., genetic risk to cancer, genetic risk to cardiac issues, etc.). The AI agent can then generate the domain-specific annotated data based on all or portions of the transcribed text data. A first dual prompt is then generated that includes a first prompt to identify identities, relationships of the identities, and clinical features of the identities, and a second prompt to generate a data object based on the domain-specific annotated data. The first dual prompt, including the first prompt and the second prompt, is inputted (e.g., sequentially) to a large language model (LLM) to generate the data object. A second dual prompt is generated that includes a third prompt to generate a normalized clinical schema, and a fourth prompt to correct inconsistencies of the data object. The second dual prompt is inputted to the LLM to generate the normalized clinical schema, and graph node data is generated based on the normalized clinical schema and in a structured format. An interactive dashboard may then be generated that displays information based on the graph node data. A user can edit the information, which may cause a regeneration of the graph node data.

In some embodiments, a system includes a non-transitory computer readable medium storing instructions, and one or more processors communicatively coupled to the non-transitory computer readable medium. The one or more processors are configured to execute the instructions to: execute an artificial intelligence agent based on a received signal identifying a domain-specific clinical context; generate, using the executed artificial intelligence agent, inquiry data characterizing an inquiry associated with the domain-specific clinical context; transmit the inquiry data to a user device; receive response data from the user device; generate, based on the inquiry data and the response data, annotated data in a structured format corresponding to the domain-specific clinical context; generate a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities; input the first dual prompt to a large language model and generate the data object; generate a second dual prompt comprising a third prompt to generate a normalized clinical schema, and a fourth prompt to correct inconsistencies of the data object; input the second dual prompt to the large language model and generate the normalized clinical schema; generate graph node data based on the normalized clinical schema, wherein the graph node data includes nodes representing the identities and edges representing the relationships of the identities; generate graphical user interface elements based on the graph node data; and transmit the graphical user interface elements for display.

In other embodiments, a computer-implemented method includes: executing an artificial intelligence agent based on a received signal identifying a domain-specific clinical context; generating, using the executed artificial intelligence agent, inquiry data characterizing an inquiry associated with the domain-specific clinical context; transmitting the inquiry data to a user device; receiving response data from the user device; generating, based on the inquiry data and the response data, annotated data in a structured format corresponding to the domain-specific clinical context; generating a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities; inputting the first dual prompt to a large language model and generate the data object; generating a second dual prompt comprising a third prompt to generate a normalized clinical schema, and a fourth prompt to correct inconsistencies of the data object; inputting the second dual prompt to the large language model and generating the normalized clinical schema; generating graph node data based on the normalized clinical schema, wherein the graph node data includes nodes representing the identities and edges representing the relationships of the identities; generating graphical user interface elements based on the graph node data; and transmitting the graphical user interface elements for display.

In yet other embodiments, a non-transitory computer readable medium has instructions stored thereon. The instructions, when executed by at least one processor, cause the at least one processor to: execute an artificial intelligence agent based on a received signal identifying a domain-specific clinical context; generate, using the executed artificial intelligence agent, inquiry data characterizing an inquiry associated with the domain-specific clinical context; transmit the inquiry data to a user device; receive response data from the user device; generate, based on the inquiry data and the response data, annotated data in a structured format corresponding to the domain-specific clinical context; generate a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities; input the first dual prompt to a large language model and generate the data object; generate a second dual prompt comprising a third prompt to generate a normalized clinical schema, and a fourth prompt to correct inconsistencies of the data object; input the second dual prompt to the large language model and generate the normalized clinical schema; generate graph node data based on the normalized clinical schema, wherein the graph node data includes nodes representing the identities and edges representing the relationships of the identities; generate graphical user interface elements based on the graph node data; and transmit the graphical user interface elements for display.

### BRIEF DESCRIPTION OF THE DRAWINGS

The features and advantages of the present invention will be more fully disclosed in, or rendered obvious by, the following detailed description of the preferred embodiments, which are to be considered together with the accompanying drawings wherein like numbers refer to like parts and further where:

FIG. 1 illustrates an example of an artificial intelligence-based clinical data generation system, in accordance with some embodiments;

FIG. 2 illustrates example components of the artificial intelligence-based clinical data generation system of FIG. 1, in accordance with some embodiments;

FIG. 3 illustrates an example data processing workflow, in accordance with some embodiments;

FIG. 4 illustrates an example graphical user interface generated by the artificial intelligence-based clinical data generation system of FIG. 1, in accordance with some embodiments;

FIG. 5 illustrates an example flowchart for generating graph node data and corresponding graphical user interface elements, in accordance with some embodiments; and

FIG. 6 illustrates an example computer system, in accordance with some embodiments.

### DETAILED DESCRIPTION

This description of the example embodiments is intended to be read in connection with the accompanying drawings that are to be considered part of the entire written description. Terms concerning data connections, coupling and the like, such as “connected” and “interconnected,” and/or “in signal communication with” refer to a relationship wherein systems or elements are electrically connected (e.g., wired, wireless, etc.) to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these example embodiments in connection with the accompanying drawings.

The embodiments described herein are directed to using artificial intelligence (AI) processes to generate, summarize, and analyze clinical data, such as clinical data related to determining genetic risk to various medical conditions. The embodiments provide scalable, AI-powered data collection technology that can facilitate risk identification and indication for genetic testing, as well as provide far more comprehensive data, thereby allowing for more efficient genetic counseling services and guidance across various medical specialties.

At least some of the embodiments described herein convert an AI voice-agent family-history phone conversation into a clinically usable, interactively editable pedigree by transcribing call audio and processing the transcript through staged intermediate data artifacts to generate a predefined family-graph data structure. The resulting graph is rendered and edited via a custom, deterministic graph-to-layout pipeline. The embodiments can include schema validation and clinical-context constraints that narrow and repair ambiguous outputs, while a deterministic visualization pipeline renders complex family structures, flags omissions and inconsistencies, and, in some instances, allows for human review and correction, ensuring that the resulting pedigree is robust and representative before being saved as part of a clinical record.

For instance, in some examples, a computer system may establish a call with a user's telephone (e.g., smartphone). The computer system can use speech-to-text processes to transcribe audio data received during the call into text, and can use text-to-speech processes to convert text data into audio data for transmission. In some examples, the computer system authenticates the user (e.g., by validating credentials and/or personal information), and can determine the domain-specific clinical context for the user based on the authentication. For instance, the user may have an established account that indicates that the user has signed on for services for a specific domain, such as genetic testing for cardiac issues. The computer system can access the account information for the user based on the authentication, and determine that the domain-specific clinical context for the user is genetic testing for cardiac issues. In some examples, the user, during the call, can select the domain-specific clinical context (e.g., by entering in a number corresponding to the domain-specific clinical context).

Once the domain-specific clinical context is determined, the computer system can execute an artificial intelligence agent based on the domain-specific clinical context. For instance, the computer system can configure an AI agent based on the domain-specific clinical context including, for instance, selecting prompts appropriate for the domain-specific clinical context that will be provided to a large language model (LLM). The computer system can input one or more of the prompts to the LLM and generate inquiry data characterizing questions related to the domain-specific clinical context. The computer system applies a text-to-speech process to the inquiry data to generate audio data, and transmits the audio data to the user's device during the call. The computer system also receives response audio data from the user's device, and applies a speech-to-text process to the response audio data to generate response text data. The computer system can input at least portions of the text data to a same or different LLM to generate follow-up questions for the user for the specific domain. The AI agent can then generate, based on the inquiry data and the response text data, annotated data in a structured format corresponding to the domain-specific clinical context.

Further, the computer system can generate a first dual prompt comprising a first prompt and a second prompt. The first prompt includes identities, relationships of the identities, and clinical features of the identities, and the second prompt includes a command to generate a data object based on the annotated data, where the data object is to identify the identities, the relationships of the identities, and the clinical features of the identities. The computer system then inputs the first dual prompt, including the first prompt and the second prompt, to the LLM and generates the data object. The data object may be in .JSON or CSV format, for example.

The computer system also generates a second dual prompt that includes a third prompt and a fourth prompt. The third prompt includes a command to generate a normalized clinical schema, and the fourth prompt includes a command to correct inconsistencies of the data object. The computer system inputs the second dual prompt, including the third prompt and the fourth prompt, to the large language model and generates the normalized clinical schema. Further, and based on the normalized clinical schema, the computer system generates graph node data (e.g., a node/edge graph) that includes, among other things, nodes representing the identities and edges representing the relationships of the identities. For instance, the nodes can represent identities such as a parent (father, mother), a child (e.g., son, daughter), uncle, aunt, grandparent, great-grandparent, niece, nephew, etc. The edges can represent the relationship between two nodes (e.g., spouses, parent-child, etc.).

In addition, the computer system can generate graphical user interface elements based on the graph node data. For example, the computer system can map the nodes and edges into a rendering engine's native types, and can apply styling rules to reflect clinical semantics (for example, adoption edges may be rendered differently from edges identifying biological relationships, and divorce edges may be distinctly styled). The computer system can then transmit the graphical user interface elements for display. For instance, the computer system can transmit the graphical user interface elements to the user's device to be displayed by an executed application (e.g., browser).

Among other advantages, the embodiments can automate otherwise manual, data collection cumbersome processes, and reduce error rates for the data collected. Moreover, the embodiments can reduce the amount of data processing resources, including processing time and processing power, that otherwise may be needed to generate clinical data. Further, the embodiments can provide a scalable, AI-powered data collection system that can facilitate risk identification and indication for genetic testing, as well as provide far more comprehensive data, thereby allowing for a more efficient evaluation of clinical data across various medical domains. Persons of ordinary skill in the art can recognize these and other advantages as well.

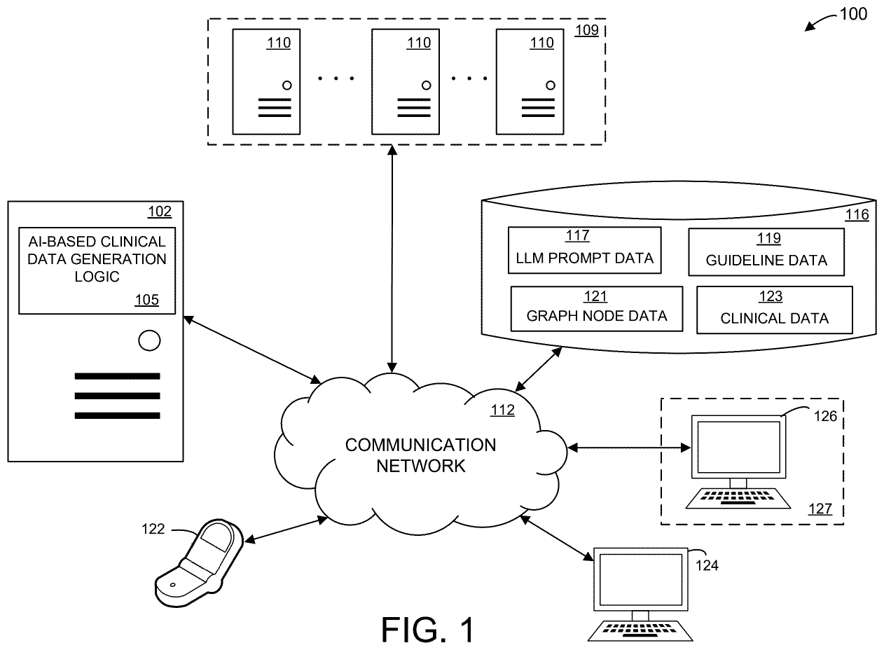

Referring now to the drawings, FIG. 1 illustrates an artificial intelligence (AI)-based clinical data generation system 100 that can, among other things, generate clinical data using artificial intelligence-based processes. The clinical data can be related to clinical genetics, for instance. As illustrated, the AI-based clinical data generation system 100 includes an AI-based clinical data (AICD) computing device 102 with AI-based clinical data generation logic 105, one or more large language model (LLM) servers 110, a plurality of user devices 122, 124, 126, and a database 116 all communicatively connected to a communication network 112.

Each of the one or more LLM servers 110 can execute one or more large language models. For instance, an LLM server 110 can host a website or portal through which LLM prompts can be provided. In response to the LLM prompts, the LLM servers 110 can input the prompts to one or more LLMs to generate output data, and the LLM servers 110 can transmit the output data in response to the received LLM prompts. Moreover, the one or more LLM servers 110 can be part of one or more cloud-based computing systems 109, for example.

In some examples, the first user device 122 and the second user device 124 can be operated by patients, while the third user device 126 can be operated by a medical professional, such as a genetic testing counselor. For instance, the third user device 126 may be associated with a genetic testing center 127 that performs genetic testing.

The database 116 can include any suitable data storage medium, such as one or more Hard Disk Drives (HDDs), one or more Solid State Drives (SSDs), one or more Non-Volatile Memories (NVMs), one or more servers, cloud-based storage, or any other suitable data repository. Each of the AICD computing device 102 and LLM servers 110 can store data to, and read data from, database 116.

Moreover, each of the AICD computing device 102, LLM servers 110, and user devices 122, 124, 126 can include one or more processors (e.g., processing units, such as graphical processing units (GPUs), central processing units (CPUs), processing cores) that can execute corresponding instructions, one or more field-programmable gate arrays (FPGAs), one or more application-specific integrated circuits (ASICs), one or more state machines, digital circuitry, and/or any other suitable circuitry. For instance, each can be a computer, a workstation, a laptop, a server, or any other suitable processing device. In addition, each of these processing devices can transmit and receive data over the communication network 112.

The communication network 112 can be, for example, a WiFi® network, a cellular network such as a 3GPP® network, a Bluetooth® network, a satellite network, a wireless local area network (LAN), a network utilizing radio-frequency (RF) communication protocols, a Near-Field Communication (NFC) network, a wireless Metropolitan Area Network (MAN) connecting multiple wireless LANs, a wide-area network (WAN), or any other suitable network. The communication network 112 may provide access to the Internet, for example.

Although FIG. 1 illustrates a single AICD computing device 102, multiple LLM servers 110, one database 116, and multiple user devices 122, 124, 126, the AI-based clinical data generation system 100 can include any number of any of these devices. In addition, the AI-based clinical data generation system 100 can include additional networked devices as well. For example, the AI-based clinical data generation system 100 can include additional computing systems, servers, and storage devices.

Moreover, although embodiments are illustrated herein having individual, discrete devices, it will be appreciated that, in some embodiments, one or more devices may be combined into a single logical and/or physical device. For example, in various embodiments, one or more of the AICD computing device 102, the LLM servers 110, and the database 116 may be combined into a single logical and/or physical device.

In some examples, the third user device 126 transmits a collect data signal to the AICD computing device 102. The collect data signal may identify a domain-specific clinical context for a patient, such as a user of the first user device 122. The AICD computing device 102 receives the collect data signal and, in response, executes an artificial intelligence (AI) agent for the domain-specific clinical context. For instance, the database 116 can store LLM prompt data 117 characterizing LLM prompts for each of a plurality of domain-specific clinical contexts (e.g., cancer, cardiac). The AICD computing device 102 can determine the domain-specific clinical context based on the received collect data signal, and receive the corresponding LLM prompt data 117 for the determined domain-specific clinical context. In some examples, the AICD computing device 102 determines, based on the received signal, one of a cardiac context and a cancer context, and configures the AI agent for execution based on the determined one of the cardiac context and the cancer context.

Further, the AICD computing device 102 can then execute the AI agent and input the LLM prompt data 117 for the determined domain-specific clinical context to an LLM. As described further herein, the LLM can be one executed locally (e.g., by the AI agent), or can be one executed by one or more of the LLM servers 110 (e.g., the AICD computing device 102 can transmit the LLM prompt data 117 to an LLM server 110 for LLM ingestion).

The LLM prompt data 117 can include a command that the LLM is a “caring and professional medical genetics receptionist that specializes in meticulously collecting detailed family histories focused on inherited cancer risk,” and a command that “while you don't provide medical advice, you leverage your medical knowledge and expertise in hereditary genetic conditions to deeply understand user responses, where your primary responsibility is to efficiently take inbound calls from patients who are scheduled for a genetic counseling appointment, and expertly guide them through a comprehensive family cancer history interview prior to their appointment.” The inputted LLM prompt data 117 can also include rules (e.g., “always collect name and date of birth,” “be concise,” “confirm exact relationships,” and “always ask for the children of aunts and uncles as well as their medical histories.”). The LLM prompt data 117 can also specify an order of questions to ask the patient for the particular domain-specific clinical context, such as “ask for name and date of birth first” and “for each person identified, ask for their cancer history.” The inputting of the LLM prompt data 117 to the LLM configures the AI agent for the particular domain-specific clinical context.

Once configured, the AICD computing device 102 generates, using the executed AI agent, inquiry data characterizing an inquiry associated with the domain-specific clinical context. The inquiry data can characterize a question such as “what is your name?”, “what is your date of birth?”, “are you ready to begin?”, and so fourth. The inquiry data can also characterize familial related questions such as “do you have any siblings?”, “what are the ages of your parents?”, and “what are the ages of your maternal grandparents?” The inquiry data can also include health related questions, such as those pertinent to the domain-specific clinical context. For instance, the inquiry data can characterize questions such as “can you tell me about your mother's health?” or “what can you share about your uncle's medical history?” The AICD computing device 102 can transmit the inquiry data to the first user device 122.

As described further herein, the first user device 122 may display the inquiry data to the patient. In some examples, the AICD computing device 102 generates audio data based on the inquiry data, and transmits the audio data to the first user device 122 to be played out of a speaker. The patient may read or hear the inquiry, and may then input (e.g., type or speak) a response. The first user device 122 may generate response data based on the patient's input. The first user device 122 may transmit the response data to the AICD computing device 102. The AICD computing device 102 can receive the response data, and the executed AI agent can input the response data to the LLM to generate follow-up inquiries. The AICD computing device 102 can save all of the inquiry data and the response data as a transcript of the information exchange (i.e., transcript data).

In response to receiving the response data, the AICD computing device 102 can generate, based on the generated inquiry data and the response data, annotated data in a structured format corresponding to the domain-specific clinical context. The annotated data characterizes a representation of the transcript, and can be generated as a structured, token-efficient representation of the transcript that reduces conversational ambiguity and filler, extracts clinically relevant facts, stabilizes identities and relationships, and imposes consistent structure. In some examples, the AICD computing device 102 generates the annotated data in structured sections, including a list of identified individuals that are assigned a corresponding identifier (e.g., P001, P002), per-person attributes required for a clinically meaningful pedigree (such as sex at birth, approximate age or date of birth, diagnoses and affected status, adoption indicators, and maternal or paternal side), and a relationship list that references individuals (e.g., spouses) by their corresponding identifiers.

Further, the AICD computing device 102 can generate a first dual prompt that includes a first prompt and a second prompt. In some examples, the AICD computing device 102 receives prompt data characterizing the first prompt and the second prompt from the LLM prompt data 117 stored in the database 116, and generates the first prompt and the second prompt based on the received prompt data. The first prompt identifies identities, relationships of the identities, and clinical features of the identities. For instance, the first prompt can indicate that the LLM is “a clinical genetics assistant trained to extract structured family history information from transcripts,” and a command to “identify all family members, their relationships to each other, and any diseases or relevant clinical features.” The second prompt can include a command to generate a data object based on the annotated data, where the data object is to identify the identities, the relationships of the identities, and the clinical features of the identities. For example, the second prompt can indicate to the LLM to “extract the following from this call transcript: (1) a list of all family members mentioned, (2) an annotated list linking each member to diseases and relationships, and (3) a natural-language summary of the overall family structure,” followed by the transcript data (i.e., the saved inquiry data and response data).

The AICD computing device 102 inputs the first dual prompt, including the first prompt and the second prompt (e.g., sequentially) to a large language model and, in response to inputting the first dual prompt, generates the data object. As described further herein, in some examples, the AICD computing device 102 transmits the first dual prompt to an LLM server 110, and the LLM server 110 inputs the first dual prompt to an LLM to generate the data object. The LLM server 110 then transmits the data object to the AICD computing device 102. As described herein, the data object can be a JSON formatted object, for instance.

The AICD computing device 102 can also generate a second dual prompt that includes a third prompt and a fourth prompt. In some examples, the AICD computing device 102 receives prompt data characterizing the third prompt and the fourth prompt from the LLM prompt data 117 stored in the database 116, and generates the third prompt and the fourth prompt based on the received prompt data. The third prompt includes a command to generate a normalized clinical schema. For example, the third prompt can indicate that the LLM is “a data-structuring engine that converts loosely structured family history JSON into a normalized medical schema,” and a command to “ensure relationship consistency, generational order, and correct sex/gender assignment.” The fourth prompt can include a command to correct inconsistencies of the data object. For instance, the fourth prompt can include a command that “given the following output from a previous extraction, correct inconsistencies and standardize the structure to this schema: {name, sex, relation_to_proband, age, diagnoses, generation}.” The fourth prompt can also include a command to “return a fully formed JSON object and a brief error log of corrections made,” such as in the example when using the JSON format. The fourth prompt can further include the data object.

The AICD computing device 102 inputs the second dual prompt, including the third prompt and the fourth prompt (e.g., sequentially) to the large language model and, in response to inputting the second dual prompt, the AICD computing device 102 generates the normalized clinical schema. As described further herein, in some examples, the AICD computing device 102 transmits the second dual prompt to an LLM server 110, and the LLM server 110 inputs the second dual prompt to an LLM to generate the normalized clinical schema. The LLM server 110 then transmits the normalized clinical schema to the AICD computing device 102. As such, the LLM can perform both schema-level validation and apply sanity-check rules to achieve graph integrity, such as enforcing a unique identifier per person, confirming referential consistency between relationships and people, ensuring parent-child directionality is valid, and preventing circular or self-referential loops, among other examples.

In some instances, the fourth prompt includes a command to generate a report that identifies any detected failures (e.g., missing identifiers, duplicate people, invalid edges, inconsistent parent sets). If the normalized clinical schema received from the LLM indicates that any failures were detected, the AICD computing device 102 re-prompts the LLM using the first dual prompt, but with an additional command to check and correct for any of the detected failures. The AICD computing device 102 then uses the re-generated data object to generate the second dual prompt as described herein, and to re-generate the normalized clinical schema. This validation and repair process can continue until no further failures are detected.

Further, the AICD computing device 102 generates graph node data 121 based on the normalized clinical schema. The graph node data 121 can include nodes representing the identities and edges representing the relationships of the identities. For example, the graph node data 121 can characterize a normalized family-tree structure with union constructs (e.g., marriage/partnership groupings) where, for instance, spousal relationships and shared children are represented in a layout-friendly manner. In other words, the generated graph node data 121 can represent lists of people and relationship groupings into one or more tree-like structures that can be traversed deterministically, while preserving the semantics required for pedigree rendering (e.g., generations, unions, siblings, multiple spouses, and disconnected components). In some examples, the AICD computing device 102 stores the generated graph node data 121 in the database 116.

In some examples, to generate the graph node data, the AICD computing device 102 receives guideline data 119 from the database 116. The guideline data 119 characterizes an algorithm and/or rules for generated graph node data 121. For instance, the guideline data 119 can characterize rules for generating union constructs, rules for applying deterministic partner ordering, rules for identifying root individuals, and rules for constructing traversal maps, among other examples.

For instance, to generate the graph node data 121, the AICD computing device 102 may execute an algorithm as defined by the guideline data 119. Based on the executed algorithm, the AICD computing device 102 selects a proband anchor when identified in the data object, or otherwise defaults to a reference individual, and constructs traversal maps from relationships including parent→children and child→parents adjacency structures. The AICD computing device 102 then assigns each connected person a “generationFromProband value”¹ using breadth-first traversal, which is later used to enforce stable generation rows in a final diagram represented by the graph node data 121. The disconnected components are handled deterministically by attempting to ¹A numerical representation of an individual's familial distance from the proband in a genetic study or pedigree. anchor them to already-assigned relatives, and otherwise defaulting any remaining nodes to a baseline generation. The AICD computing device 102 can also apply deterministic partner ordering in two-parent relationships when sex is known, so spouse placement remains consistent. Once this normalization process is complete, the AICD computing device 102 converts the data object into one or more hierarchical trees by identifying root individuals (those with no parents) and recursively building a person-centric structure that attaches direct children and groups partner and any shared children under explicit union/marriage constructs. During this construction the AICD computing device 102 tracks processed individuals and processed unions to avoid duplication and cycles, and also applies targeted normalization for proband-parent unions and marriage ordering so that clinically central structures remain stable and predictable in the resulting family-tree representation, i.e., the graph node data 121.

Further, and as described herein, the AICD computing device 102 can generate graphical user interface elements based on the graph node data 121. The graphical user interface elements can characterize a dashboard that displays information based on the graph node data 121. For instance, the dashboard can display the identities, the relationships between the identities, attributes of the identities, and any health related conditions by any of the identities, such as those related to the particular domain-specific clinical context, as identified by the graph node data 121, among other things. The AICD computing device 102 can transmit the graphical user interface elements for display. For instance, the AICD computing device 102 can transmit the graphical user interface elements to the first user device 122 to display the dashboard. In some examples, the transmission is performed through secure channels. For instance, the graphical user elements may be encrypted using a public key, and decrypted by the first user device 122 using a corresponding private key. In all cases, qualifying health-related information is stored, manipulated, and transmitted in accordance with Health Insurance Portability and Accountability Act (HIPAA) regulations.

In some examples, the dashboard allows a user to edit information (e.g., a name, date of birth, child assignment, etc.). Based on any edits provided, the AICD computing device 102 can automatically update the response data, and regenerate the graph node data 121 based on the updated response data, as described above.

In some examples, the AICD computing device 102 can generate a fifth prompt that includes a request to output a clinician-friendly summary note that summarizes pedigree and risk findings based on the provided graph node data 121. The clinical-friendly summary note can include, for instance, clinician-facing family-history summaries, structured reporting fields, and computed risk outputs (e.g., risk scoring based on pedigree structure and diagnoses). In some examples, the AICD computing device 102 receives prompt data characterizing the fifth prompt from the LLM prompt data 117 stored in the database 116, and generates the fifth prompt based on the received prompt data. The AICD computing device 102 inputs the fifth prompt and the graph node data 121 to the LLM and generates clinical data 123, and stores the clinical data 123 in the database. The AICD computing device 102 can also generate graphical user interface elements based on the clinical data 123, and transmit the graphical user interface elements to a device, such as to third user device 126.

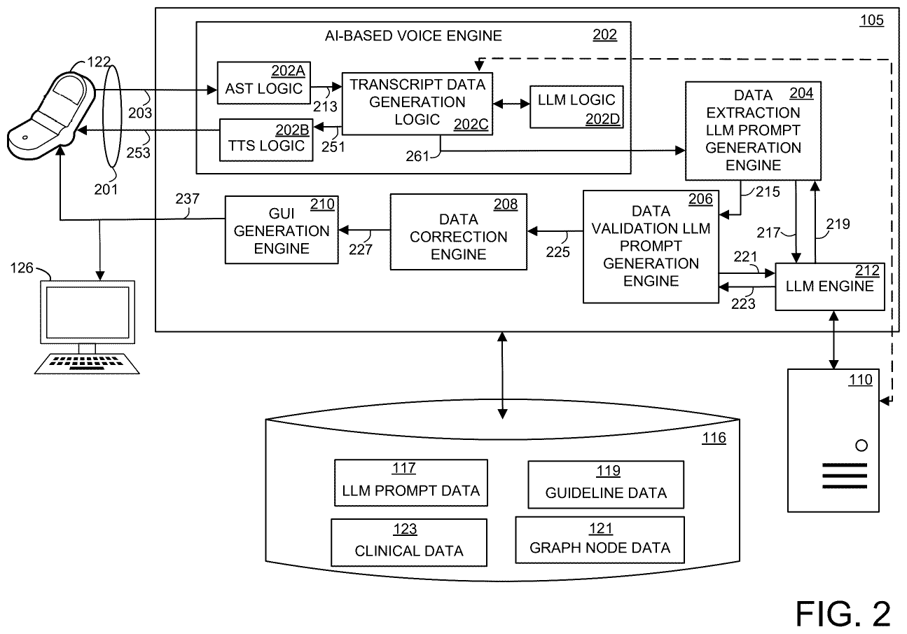

FIG. 2 illustrates example components of the AI-based clinical data generation logic 105 of the AICD computing device 102. As illustrated, the AI-based clinical data generation logic 105 includes AI-based voice engine 202, data extraction LLM prompt generation engine 204, data validation LLM prompt generation engine 206, data correction engine 208, GUI generation engine 210, and LLM engine 212. Each of the AI-based voice engine 202, data extraction LLM prompt generation engine 204, data validation LLM prompt generation engine 206, data correction engine 208, GUI generation engine 210, and LLM engine 212 can be implemented by any suitable hardware or hardware and software combination. For example, the AI-based voice engine 202, data extraction LLM prompt generation engine 204, data validation LLM prompt generation engine 206, data correction engine 208, GUI generation engine 210, and LLM engine 212 can be implemented, in whole or in part, by any combination of: one or more processors executing instructions; one or more FPGAs; one or more ASICs; one or more state machines; digital circuitry; and/or any other suitable circuitry.

As illustrated, the AI-based voice engine 202 and the first user device 122 can engage in a voice call 201. To facilitate audio communications, the AI-based voice engine 202 includes advanced speech-to-text (AST) logic 202A, text-to-speech (TTS) logic 202B, transcript data generation logic 202C, and LLM logic 202D. The AST logic 202A can apply a speech-to-text process (e.g., speech-to-text algorithm) to incoming audio data 203 to generate incoming text data 213. The TTS logic 202B can apply a text-to-speech process (e.g., text-to-speech algorithm) to outgoing text data 251 to generate outgoing audio data 253.

To generate outgoing text data 251, the transcript data generation logic 202C can input prompts to the LLM logic 202D. The LLM logic 202D inputs the received prompts to an executed LLM to generate corresponding output data, and transmits the generated output data to the transcript data generation logic 202C. In some examples, the transcript data generation logic 202C transmits the prompts to an LLM server 110, and receives the generated output data from the LLM server 110. In some examples, the transcript data generation logic 202C generates inquiry data characterizing an inquiry associated with a domain-specific clinical context based on the received output data, and packages the inquiry data into outgoing text data 251 packets that are formatted according to a protocol established between the TTS logic 202B and the transcript data generation logic 202C. The transcript data generation logic 202C then transmits the outgoing text data 251 packets to the TTS logic 202B.

In some examples, the transcript data generation logic 202C generates a configuration prompt that includes a command indicating that the LLM is collecting health information of a domain-specific clinical context for a patient, and a list of questions that need to be asked. The transcript data generation logic 202C transmits the configuration prompt to the LLM logic 202D, and the LLM logic 202D inputs the configuration prompt to the LLM. Once configured, the AI-based voice engine 202 and the first user device 122 can engage in a conversation during the voice call 201. For instance, during the call, the transcript data generation logic 202C can generate conversation prompts based on the incoming text data 213. The transcript data generation logic 202C can also transmit the conversation prompts to the LLM logic 202D to generate corresponding LLM output data, and can generate outgoing text data 251 based on the LLM output data received from the LLM logic 202D.

Further, during the call, the AI-based voice engine 202 can generate domain-specific annotated data 261 based on the incoming text data 213 and output data received from the LLM logic 202D. Once the call is complete, the AI-based voice engine 202 can transmit the domain-specific annotated data 261, which characterizes the entire conversation, to the data extraction LLM prompt generation engine 204.

The data extraction LLM prompt generation engine 204 can generate a first dual prompt 217 that includes a first prompt and a second prompt. As described herein, the first prompt can identify identities, relationships of the identities, and clinical features of the identities, and the second prompt can include a command to generate a data object based on the provided domain-specific annotated data 261, where the data object identifies the identities, the relationships of the identities, and the clinical features of the identities. The data extraction LLM prompt generation engine 204 can further generate the first dual prompt 217 to include the domain-specific annotated data 261. The data extraction LLM prompt generation engine 204 can transmit the first dual prompt 217 to the LLM engine 212, and the LLM engine 212 inputs the first dual prompt 217 to an LLM to generate a data object 219, such as a JSON data object. The data extraction LLM prompt generation engine 204 packages the data object 219 within a first stage data object message 215, and transmits the first stage data object message 215 to the data validation LLM prompt generation engine 206.

The data validation LLM prompt generation engine 206 receives the first stage data object message 215, and extracts the data object 219. Further, the data validation LLM prompt generation engine 206 generates a second dual prompt 221 that includes a third prompt and a fourth prompt. As described herein, the third prompt can include a command to generate a normalized clinical schema, and the fourth prompt can include a command to correct inconsistencies of the provided data object 219. The data extraction LLM prompt generation engine 204 can further generate the second dual prompt 221 to the data object 219. The data validation LLM prompt generation engine 206 can transmit the second dual prompt 221 to the LLM engine 212, and the LLM engine 212 inputs the second dual prompt 221 to the LLM to generate the normalized clinical schema 223. The data validation LLM prompt generation engine 206 packages the normalized clinical schema 223 within a second stage data object message 225, and transmits the second stage data object message 225 to the data correction engine 208.

The data correction engine 208 can receive the second stage data object message 225, and can extract the normalized clinical schema 223 from the second stage data object message 225. Further, the data correction engine 208 can generate graph node data 227 based on the normalized clinical schema based on any of the processes described herein. The graph node data 227 can characterize a graph that includes nodes representing the identities and edges connecting pairs of nodes, where the edges represent a relation between the two nodes. The data correction engine 208 can transmit the graph node data 227 to the GUI generation engine 210.

The GUI generation engine 210 can receive the graph node data 227, and generate graphical user interface elements 237 based on the graph node data 227. As described further herein, the graphical user interface elements 237 can characterize an interactive dashboard that displays information based on the graph node data 227. For instance, in some examples, the GUI generation engine 210 can generate the graphical user interface elements 237 to characterize a clinician-facing dashboard, and can transmit the graphical user interface elements 237 to the third user device 126 for display to a clinician. In some examples, the dashboard allows the clinician to edit the information. The inputted edits may cause the third user device 126 to transmit data characterizing the changes to the AI-based clinical data generation logic 105, and the AI-based clinical data generation logic 105 may regenerate the graph node data 227 based on the received changes.

In some examples, the GUI generation engine 210 can generate the graphical user interface elements 237 to characterize a patient-facing dashboard, and can transmit the graphical user interface elements 237 to the first user device 122 for display to a patient. In some examples, the dashboard allows the patient to edit certain information. The inputted edits may cause the first user device 122 to transmit data characterizing the changes to the AI-based clinical data generation logic 105, and the AI-based clinical data generation logic 105 may regenerate the graph node data 227 based on the received changes.

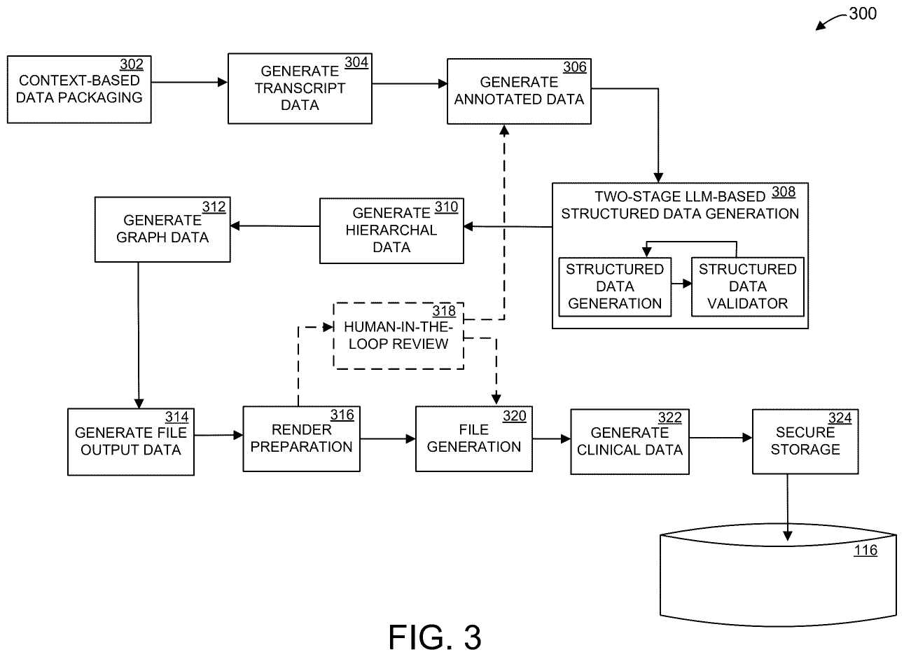

FIG. 3 illustrates a data processing workflow 300 that can be carried out by the AICD computing device 102, for example. As illustrated, the data processing workflow 300 includes various data processing stages.

Beginning at the context-based data packaging stage 302, an AI-based voice agent is configured using a domain-specific clinical context package selected from a curated context store (e.g., database 116). The AI-based voice agent can establish a call, and can convert digital data to audio data for transmission, as well as receive audio data and convert the received audio data to digital data, as described further herein. Further, the context store is a structured, clinically maintained knowledge base that organizes guideline-grounded interviewing objectives, required data elements, definitions and exclusions, escalation and safety guardrails, and disambiguation strategies into reusable, versioned packages aligned to specific clinical focuses (for example, cardiac history versus cancer history). This stage standardizes topic ordering, ensuring coverage of at least the minimum required fields for downstream pedigree construction, and triggers targeted clarification when ambiguity arises (such as when multiple relatives share the same name or the direction of a relationship is unclear). Packages are keyed by disease focus and can also be scoped by organization or workflow, so the active agent inherits the appropriate constraints and clinical requirements for that setting. The AI-based voice agent can store converted audio data as response data, and can additionally enable real-time tools during the call to confirm or restate schema-critical entities as they are captured—such such as the proband, first-degree relatives, diagnoses, and ages of onset—ensuring the resulting transcript and call artifacts are complete and consistent. The output of this stage is a domain-shaped interview record optimized for reliable downstream extraction, graph construction, and clinical review.

After the call concludes, the data processing workflow 300 proceeds to the generate transcript data state 304, where the voice-provider call events are converted into a canonical call record that becomes the system's source of truth for all downstream processing. The ingestion flow begins when a webhook notifies a backend (e.g., a backend processing unit of the AICD computing device 102) that the call has completed. The webhook request can be authenticated using a shared secret header to prevent unauthorized submissions. Once validated, the backend retrieves the complete set of call artifacts from the voice provider, including the transcript, timestamps, call metadata (such as direction, start and end times, and end reason), and any provider-side structured data fields. These provider payloads are normalized into consistent internal fields and persisted as a call record in the database 116, ensuring that extraction stages operate on a stable artifact rather than transient webhook content. In some examples, telephony routing identifiers (such as a phone number or line identifier) are also mapped to an organization to auto-populate organization scope for access control and downstream routing. This stage produces a secure, auditable ingestion boundary: the transcript and metadata are captured once, stored reliably (e.g., as transcript data), and made available for staged extraction, in some examples human review, and pedigree generation.

At the generate annotated data stage 306, an intermediate compressed representation of the transcript is generated, referred to herein as annotated data. The annotated data can be a structured, token-efficient representation of the interview that reduces conversational ambiguity and filler, extracts clinically relevant facts, stabilizes identities and relationships, and imposes consistent structure. This improves the reliability of the subsequent schema-conformant JSON family graph required for deterministic layout rendering and, in some examples, human review. This stage extracts and normalizes the key entities and facts characterized by the annotated data into a staged artifact optimized for downstream processing. In practice, the annotated data is organized into structured sections, including a roster of individuals assigned stable internal identifiers (e.g., P001, P002), per-person attributes required for a clinically meaningful pedigree (such as sex at birth, approximate age or date of birth, diagnoses and affected status, adoption indicators, and maternal or paternal side), and an explicit relationship list that references individuals by identifier rather than name alone. The prompt templates, model identifier, and generated annotated output are stored alongside the call record as an intermediate artifact, creating a traceable extraction workspace that supports auditability and benchmarking as prompts or models evolve.

The data processing workflow 300 then reaches the two-stage LLM-based structured data generation stage 308 where the intermediate representation characterized by the annotated data is converted to, in this example, a single pedigree JSON family graph through a two-stage LLM-based pipeline (e.g., structured data generation and structured data validation) designed to produce a structurally reliable artifact for storage, visualization, and export. In the first stage (e.g., structured data generation), a dedicated prompt template maps the annotated data into a predefined, typed family-graph schema (for example, a payload containing people[] and relationships[]), so the output is constrained to a schema-conformant JSON structure. For instance, as described further herein, a first dual prompt is generated that includes a first prompt to identify identities, relationships of the identities, and clinical features of the identities, and a second prompt to generate a JSON data object based on the annotated data, wherein the data object identifies the identities, the relationships of the identities, and the clinical features of the identities. The first dual prompt is then input to an LLM to generate the JSON data object.

In the second stage (e.g., structured data validation), the generated JSON data object is passed through an LLM-based deterministic parser and validator that performs both schema-level validation and additional sanity-check rules for graph integrity, such as enforcing a unique identifier per person, confirming referential consistency between relationships and people, ensuring parent-child directionality is valid, and preventing circular or self-referential loops. While the validation may be deterministic using, for example, schema and/or rule checks, an LLM produces candidate outputs where, as described further herein, the LLM can be re-prompted to repair any validation failures. For instance, as described further herein, a second dual prompt is generated that includes a third prompt to generate a normalized clinical schema, and a fourth prompt to correct inconsistencies of the JSON data object. The second dual prompt is then input to an LLM to generate the normalized clinical schema.

In some examples, when the LLM-based validator (e.g., during structured data validation) detects issues, it outputs a structured report describing the failures (e.g., missing identifiers, duplicate people, invalid edges, inconsistent parent sets), and the report is provided as feedback to automatically re-prompt the first stage generation step to repair the JSON data object. This generate-validate-repair loop can continue until the pedigree JSON data object passes all checks, at which point the validated graph is persisted as the canonical structured output, and stored in the database (e.g., database 116) as a pedigree_json data object for downstream rendering and, in some examples, human review. In some instances, prompt templates and any model identifiers used at each stage are also stored in the database 116 for auditability and benchmarking.

At the generate hierarchical data stage 310, the pedigree_json data object is converted into hierarchical data characterizing a family-tree structure that is explicitly hierarchical (parent→child) and introduces explicit union constructs (e.g., marriage/partnership groupings) so that spouse relationships and shared children can be represented in a way that is layout-friendly. This stage is essentially a normalization pass that turns “lists of people and relationship groupings” into one or more tree-like structures that can be traversed deterministically, while preserving the semantics required for pedigree rendering (generations, unions, siblings, multiple spouses, and disconnected components). In some examples, a proband anchor is selected when present; otherwise a reference individual is defaulted to. Traversal maps are then constructed from relationships[], including parent→children and child→parents adjacency structures. Each connected component (e.g., connected person) is then assigned a generationFromProband value using breadth-first traversal, which is later used to enforce stable generation rows in the final diagram. Moreover, disconnected components are handled deterministically by attempting to anchor them to already-assigned relatives. Otherwise, disconnected components are defaulted to a baseline generation. The pipeline can also apply deterministic partner ordering in two-parent relationships when sex is known, so spouse placement remains consistent across renders. Once normalization is complete, the algorithm converts the flat graph into one or more hierarchical trees by identifying root individuals (those with no parents) and recursively building a person-centric structure that attaches direct children and groups partner+shared children under explicit union/marriage constructs. During this construction processed individuals and processed unions are tracked to avoid duplication and cycles, and targeted normalization is applied for proband-parent unions and marriage ordering so that clinically central structures remain stable and predictable in the resulting family-tree representation.

At the generate file output data stage 314, a render graph representation (e.g., graph_json) of the hierarchical data is generated. The render graph representation consists of explicit nodes and edges designed for a canvas-based rendering engine. This step takes the tree representation and emits render primitives such as person nodes, synthetic union (marriage) nodes, parent-child edges, and spouse edges. Disconnected components are unified under a hidden root for layout computation so the rendering engine can perform a single deterministic pass, while spouse relationships are tracked separately from parent-child links so they can be rendered and styled with distinct semantics. The output of this transformation is a consistent node/edge graph, referred to herein as graph node data (e.g., graph node data 121), that is purpose-built for layout, styling, and interaction, rather than a storage schema. In some examples, the generated graph node data (e.g., graph node data 121) is stored in a database (e.g., database 116).

At the render preparation stage 316, a deterministic layout is determined and post-processing rules are applied to the render graph representation (e.g., graph_json) that stabilize pedigree readability across re-renders. Positions may be first computed using a tree layout algorithm, and then vertical placement is normalized using generationFromProband so that each generation (e.g., great-grandparent generation, grandparent generation, parent generation, child generation, etc.) aligns to a consistent row regardless of input order. In some examples, union nodes are centered relative to linked parents and children, and spacing adjustments are applied for multi-spouse structures to reduce overlap. In addition, duplicative render nodes are identified and removed, such as when the same person appears in multiple structural contexts (e.g., as both a main node and a spouse-positioned node), by using deterministic precedence rules (including special handling for proband-parent placements). Finally, nodes and edges are mapped into the rendering engine's native types, with styling rules applied to reflect clinical semantics (for example, adoption edges rendered differently from biological relationships, and divorce edges styled distinctly). The renderer can also control viewport fitting and update strategy (e.g., replacing node arrays rather than merging) to avoid positional drift and ensure consistent interactive behavior.

In some examples, user actions (add parent/child/spouse/sibling, edit attributes, delete subtrees) are implemented as constrained operations on the underlying people[] and relationships[] state, and each edit can trigger an onChange (people, relationships) update so the canonical pedigree_json data object remains the single source of truth. The updated pedigree_json data object is then re-run through the same transformation pipeline described in the above data processing stages so the visual representation stays consistent with the persisted graph, enabling interactive correction without breaking genealogical integrity.

In some examples, as illustrated by the human-in-the-loop review stage 318, after the validated pedigree_json data object has been transformed into a rendered, editable diagram, a human-in-the-loop review phase can be entered. This stage can ensure that the pedigree is clinically meaningful before it is finalized and persisted as the canonical record. For instance, this stage can allow for the correction of ambiguities and omissions that may escape the automated parser such as, for instance, multiple relatives sharing the same name, uncertain relationship direction, incomplete parent information, or conflicting statements across different parts of the call. In this stage, a dashboard, such as graphical user interface 401, presents the transcript and call artifacts alongside the interactive pedigree so that a reviewer can interpret the visual structure in context and compare it directly against the source conversation. The dashboard allows reviewers to identify missing family members, correct incorrectly linked relationships, reconcile duplicates, and complete clinical annotations that are necessary for downstream use. During review, the dashboard exposes constrained editing actions that allows for editing the underlying graph state (e.g., the canonical people[] and relationships[] in pedigree_json data object). For instance, reviewers can add or modify individuals, parents, children, spouses, and siblings. The reviewers can also correct unions and parent-child relationships, update person-level clinical attributes, and remove nodes or subtrees where appropriate. Each action can update the pedigree_json data object as the single source of truth, and the visualization is re-derived through the same deterministic transformation pipeline so that the diagram remains consistent and structurally valid after each change. For instance, as illustrated, for each change, the data processing workflow 300 may proceed back to the generate annotated data stage 306 to regenerate the pedigree_json data object. When the reviewer is satisfied that the pedigree matches clinical intent, the finalized pedigree_json data object is saved (e.g., in database 116 as graph node data 121) as the approved state and proceeds to persist it as the canonical pedigree record, preserving traceability back to the original call and intermediate extraction artifacts. This representation is intentionally independent of the visualization layer: the layout engine is a deterministic function of pedigree_json data object, meaning the same underlying graph can be re-rendered consistently without storing any layout-specific state. As a result, once the reviewed pedigree_json data object is saved, it becomes a stable, reusable source of truth that can be transformed into interoperable formats without any reliance on large language models.

At the file generation stage 320, deterministic conversion logic is used to map people[] and relationships[] into standardized outputs-such as a CSV file (e.g., PED-compatible CSV file), by deriving parent identifiers from parent-child edges and deriving partner links from co-parent/union structures. The resulting export content can be stored alongside the pedigree record (e.g., graph node data 121) for immediate download and, if it is missing, can be regenerated on demand from the same canonical pedigree_json data object, ensuring consistent interoperability with downstream clinical systems.

Further, at the generate clinical data stage 322, clinical data, such as clinical data 123, can be generated based on the export content and/or the pedigree record (e.g., the graph node data 121). As described herein, the clinical data can be generated by inputting, to an LLM, a prompt (e.g., the fifth prompt) that includes a request to output a clinician-friendly summary note that summarizes pedigree and risk findings based on the provided graph node data, where the clinical data is derived, at least in part, from the output of the LLM. The clinical data can include clinician-facing family-history summaries, structured reporting fields, and computed risk outputs (for example, risk scoring based on pedigree structure and diagnoses). These clinical outputs may be produced using a combination of deterministic rules, clinical calculators, or probabilistic models depending on the artifact type and clinical domain. Moreover, the clinical outputs can be regenerated consistently and traced back to the same reviewed pedigree_json data object.

Further, at the secure storage stage 324, the pedigree_json data object and the clinical data can be securely stored in a database, such as database 116. For instance, each of the pedigree_json data object and the clinical data may be encrypted prior to storage. Moreover, the pedigree_json data object and the clinical data are stored in accordance with all HIPAA requirements.

Across every stage of the data processing workflow 300, call artifacts and derived pedigree outputs can be processed as governed clinical data and security and traceability for this data can be enforced accordingly. For instance, at ingestion, webhook requests from the voice provider can be authenticated using a shared secret to prevent unauthorized or replayed submissions from creating or modifying call records. For application users, access to calls, pedigrees, and exports is controlled through authenticated requests (e.g., JWT verification) combined with role-based authorization, ensuring that only permitted roles can initiate extraction, review, edit, or persist clinical pedigree artifacts. Further, records can be scoped to an organization, so non-privileged users can only access data associated with their organization; telephony routing identifiers (such as a phone number or line identifier) can be mapped to an organization during ingestion to automatically apply correct scoping and reduce misrouting of sensitive data. In addition to access control, provenance for the artifacts created from each call can be preserved so that outputs can be audited, reproduced, and compared over time. Intermediate and final products-including annotated data, the validated pedigree JSON, derived exports, and derived clinical reports—can be stored alongside the parameters used to generate them (for example, prompt templates, model identifiers, and versioned clinical context packages). This provenance layer enables repeatability and benchmarking as prompts, models, and clinical guidance evolve, and it provides an explicit trace from the saved pedigree back to the source transcript and the intermediate transformations that produced it.

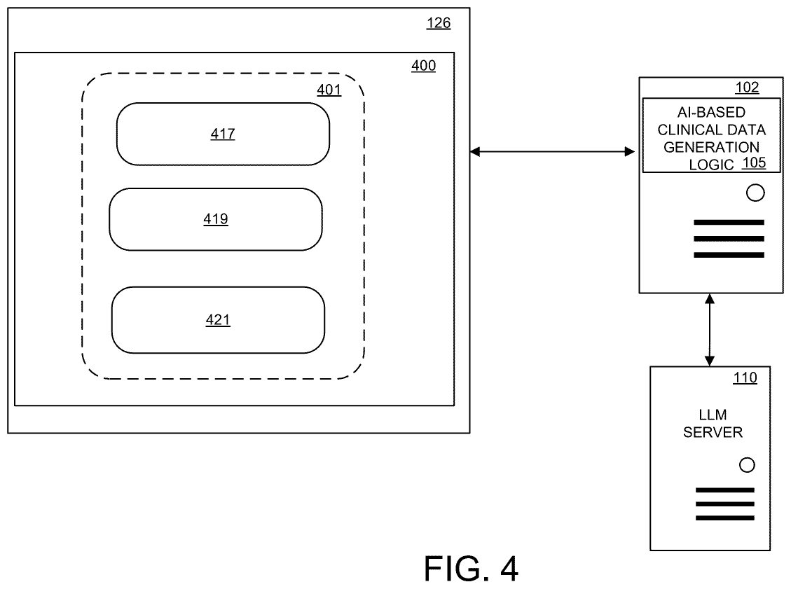

FIG. 4 illustrates a graphical user interface 401 that can be displayed by the third user device 126 on a display 400. As illustrated, the graphical user interface 401 includes a first display portion 417, a second display portion 419, and a third display portion 421. The graphical user interface 401 (e.g., dashboard) may be generated by the AICD computing device 102 as described further herein.

For instance, the AICD computing device 102 can execute an artificial intelligence agent to generate domain-specific annotated data. The AICD computing device 102 can generate a first dual prompt that includes a first prompt to identify identities, relationships of the identities, and clinical features of the identities, and a second prompt to generate a data object based on the clinical data. The AICD computing device 102 transmits the first dual prompt to the LLM server 110, and the LLM server 110 inputs the first dual prompt to an LLM to generate the data object. The LLM server 110 then transmits the data object to the AICD computing device 102. The AICD computing device 102 can also generate a second dual prompt that includes a third prompt to generate a normalized clinical schema, and a fourth prompt to correct inconsistencies of the data object. The AICD computing device 102 transmits the second dual prompt to the LLM server 110, and the LLM server 110 inputs the second dual prompt to the LLM to generate the normalized clinical schema. The LLM server 110 then transmits the normalized clinical schema to the AICD computing device 102. Further, the AICD computing device 102 can generate graph node data based on the normalized clinical schema. The AICD computing device 102 can then generate graphical user interface elements characterizing the graphical user interface 401 based on the graph node data, and transmit the graphical user interface elements to the third user device 126 for displaying the graphical user interface 401 on the display 400.

In some examples, the AICD computing device 102 generates the graphical user interface elements to characterize a graphical user interface 401 that presents assessment progress, results, and next-step actions within a controlled workflow interface. For instance, the first display portion 417 may display assessment progress, the second display portion 419 may display results, and the third display portion 421 may display next-step actions.

The graphical user interface 401 operates as a state-aware presentation layer that reflects backend assessment status rather than static content. The assessment is modeled as a multi-stage workflow comprising discrete states such as intake completion, automated analysis, results preparation, and readiness for clinical follow-up. These states can be represented by machine-readable status indicators persisted with the patient record and updated automatically in response to backend events, including the completion of transcript processing, eligibility, evaluation, or clinician review.

The graphical user interface 401 can dynamically render progress indicators based on the current workflow state, visually communicating completed, active, and pending stages of the assessment. Content visibility and patient interaction capabilities are conditionally gated based on backend release conditions, such that assessment results and recommendations are withheld until predefined criteria are satisfied. These criteria may include completion of automated analysis, clinician or genetic counselor review, or explicit authorization to release report artifacts. While results are gated, the graphical user interface 401 can present an intermediate placeholder state indicating that analysis is ongoing or pending review without disclosing unvalidated clinical conclusions.

Once release conditions are satisfied, the AICD computing device 102 can update the graphical user interface 401 with patient-facing next-step content, which can be presented within a dedicated portion of the graphical user interface 401 reserved for personalized action items derived from eligibility and clinical findings, along with explanatory context and associated timelines. The graphical user interface 401 may further provide interaction controls enabling the patient to initiate or defer follow-up actions, including requesting contact with a clinician, initiating a phone call, or scheduling communication for a later time. The availability and behavior of these controls can be governed by a backend workflow state and organizational configuration, ensuring that patient actions are enabled only when clinically and operationally appropriate. For instance, the patient graphical user interface 401 views can be derived from an authoritative backend state rather than client-side inference. The AICD computing device 102 can update the graphical user interface 401 to reflect assessment status, gating flags, report availability, and next-step content from secure backend endpoints, ensuring synchronization with the underlying clinical workflow. The graphical user interface 401 can function as an orchestration and presentation layer that consumes canonical pedigree data, guideline eligibility outputs, and generated report artifacts, thereby maintaining consistency across clinician workflows, patient communication, and persisted clinical records while preserving auditability throughout the assessment lifecycle.

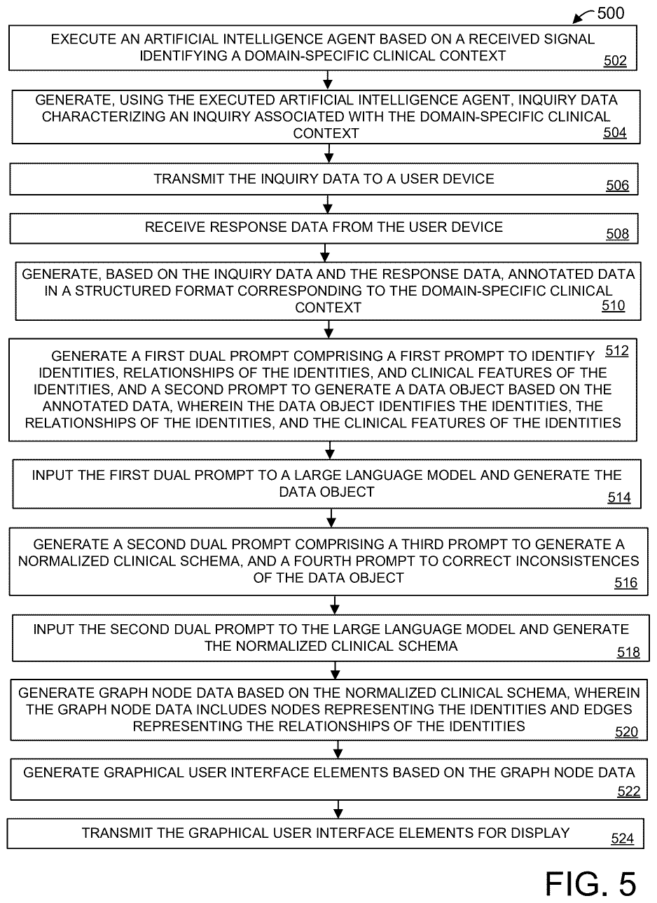

FIG. 5 is a flowchart of a method 500 for generating graph node data (e.g., graph node data 121), as well as graphical user interface elements based on the graph node data. The example method 500 can be carried out by any suitable processing device with AI-based clinical data generation logic 105, such as the AICD computing device 102 described herein.

Beginning at block 502, an artificial intelligence agent is executed based on a received signal identifying a domain-specific clinical context. Further, at block 504, inquiry data is generated using the executed artificial intelligence agent. The inquiry data characterizes an inquiry associated with the domain-specific clinical context. At block 506, the inquiry data is transmitted to a user device. Additionally, at block 508, response data is received from the user device. Further, at block 510, based on the inquiry data and the response data, annotated data is generated in a structured format corresponding to the domain-specific clinical context.

Proceeding to block 512, a first dual prompt is generated. The first dual prompt comprises a first prompt to identify identities, relationships of the identities, and clinical features of the identities, and a second prompt to generate a data object based on the annotated data, where the data object identifies the identities, the relationships of the identities, and the clinical features of the identities. At block 514, the first dual prompt is inputted to a large language model and the data object is generated.

Further, at block 516, a second dual prompt is generated. The second dual prompt comprises a third prompt to generate a normalized clinical schema, and a fourth prompt to correct inconsistencies of the data object. At block 518, the second dual prompt is inputted to the large language model and the normalized clinical schema is generated.

Proceeding to block 520, graph node data is generated. The graph node data is generated based on the normalized clinical schema, and includes nodes representing the identities and edges representing the relationships of the identities. At block 522, graphical user interface elements are generated based on the graph node data. Further, at block 524, the graphical user interface elements are transmitted for display.

Although the methods described above are with reference to the illustrated flowcharts, it will be appreciated that many other ways of performing the acts associated with the methods can be used. For example, the order of some operations may be changed, and some of the operations described may be optional.

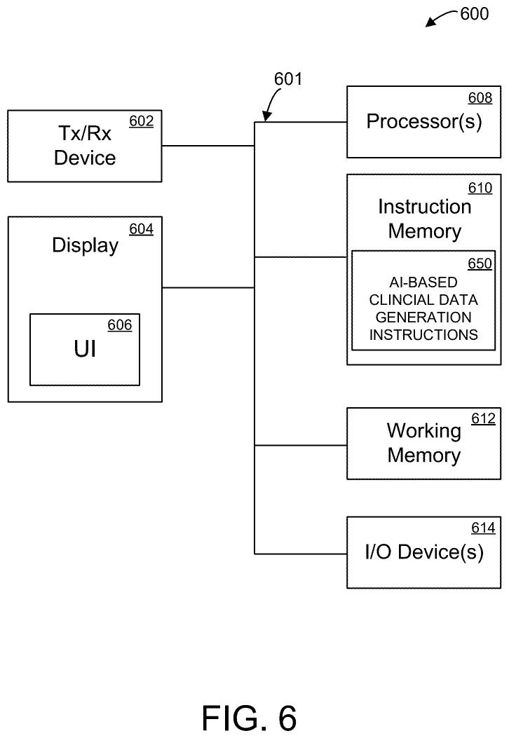

FIG. 6 illustrates a block diagram of a computing device 600, in accordance with some embodiments. Although FIG. 6 is described with respect to certain components shown therein, it will be appreciated that the elements of the computing device 600 may be combined, omitted, and/or replicated. In addition, it will be appreciated that additional elements other than those illustrated in FIG. 6 may be added to the computing device 600.

As shown in FIG. 6, the computing device 600 may include one or more processors 608, instruction memory 610, working memory 612, one or more input/output devices 614, transceiver 602, and display 604 with user interface 606, each operatively coupled to one or more data buses 601. The data buses 601 allow for communication among the various devices, and can include wired, or wireless, communication channels.

The one or more processors 608 may include any processing circuitry operable to control operations of the computing device 600. In some embodiments, the one or more processors 608 include one or more distinct processors, each having one or more cores (e.g., processing circuits). Moreover, each of the distinct processors may have the same or different structure. For instance, the one or more processors 608 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, a very long instruction word (VLIW) microprocessor, and/or other processing device. The one or more processors 608 may also be implemented by a controller, a microcontroller, an application specific integrated circuit (ASIC), a field programmable gate array (FPGA), or a programmable logic device (PLD), for example.

In some embodiments, the one or more processors 608 can implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, and user interaction applications.

The instruction memory 610 may store instructions that are accessed (e.g., read) and executed by at least one of the one or more processors 608. For example, the instruction memory 610 may be a non-transitory, computer-readable storage medium such as a read-only memory (ROM), an electrically erasable programmable read-only memory (EEPROM), flash memory (e.g., NOR and/or NAND flash memory), content-addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 608 may perform a certain function or operation by executing code, stored on the instruction memory 610, embodying the function or operation. For example, the one or more processors 608 may execute code stored in the instruction memory 610 to perform one or more of any function, method, or operation disclosed herein, such as by executing AI-based clinical data generation instructions 650 to perform the one or more of the operations described with respect to AI-based clinical data generation logic 105, AI-based voice engine 202, data extraction LLM prompt generation engine 204, data validation LLM prompt generation engine 206, data correction engine 208, GUI generation engine 210, and LLM engine 212.

Additionally, the one or more processors 608 may store data to, and read data from, the working memory 612. For example, the one or processors 608 may store a working set of instructions to the working memory 612, such as instructions (e.g., AI-based clinical data generation instructions 650) loaded from the instruction memory 610. The one or more processors 608 may also use the working memory 612 to store dynamic data created during one or more operations. The working memory 612 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g., NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separated instruction memory 610 and working memory 612, it will be appreciated that the computing device 600 may include a single memory unit that operates as both instruction memory and working memory.

In some embodiments, the instruction memory 610 and/or the working memory 612 includes an instruction set in the form of a file for executing various methods, such as for any of the methods described herein. The instruction set may be stored in any acceptable form of computer readable instructions, including source code or various appropriate programming languages. Some examples of programming languages that may be used to store the instruction set include, but are not limited to: Java, JavaScript, C, C++, C #, Python, Objective-C, Visual Basic, .NET, XML, CSS, SQL, NoSQL, Rust, Perl, and assembly. In some embodiments, a compiler or interpreter converts the instruction set into machine executable code for execution by the one or more processors 608.

The input/output devices 614 may include any suitable device that allows for data input or output. For example, the input/output devices 614 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 602 can allow for communication with a network, such as communication network 112. For example, if a communication network is a cellular network, the transceiver 602 can allow communications with the cellular network. The one or more processors 608 are operable to receive data from, or send data to, a network, via the transceiver 602.

The display 604 may include a screen such as, for example, a Liquid Crystal Display (LCD) screen, a touchscreen, a light-emitting diode (LED) screen, an organic LED (OLED) screen, a movable display, or a projection device. In some embodiments, the display 604 may include a coder/decoder, also known as Codecs, to convert digital media data into analog signals. For example, the display 604 may include video Codecs, audio Codecs, or any other suitable type of Codec.

In some examples, the display 604 may display the user interface 606 (e.g. via a touchscreen). The user interface 606 may enable user interaction. For example, the user interface 606 may allow a user to interact with an application. In some embodiments, a user may interact with the user interface 606 by engaging one or more of the input/output devices 614. In some embodiments, the display 604 may be a touchscreen, where the user interface 606 is displayed on the touchscreen.

In some embodiments, the computing device 600 may be a computer, a workstation, a laptop, a server such as a cloud-based server, or any other suitable device. In some embodiments, the computing device 600 is a server that includes one or more processing units, such as one or more graphical processing units (GPUs), one or more central processing units (CPUs), and/or one or more processing cores. The computing device 600 may, in some embodiments, execute one or more virtual machines.

The methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that the computer becomes a special purpose computer for practicing the methods. When implemented on a general-purpose processor, the computer program code segments configure the processor to create specific logic circuits. The methods may alternatively be at least partially embodied in application-specific integrated circuits for performing the methods.

The foregoing is provided for purposes of illustrating, explaining, and describing embodiments of these disclosures. Modifications and adaptations to these embodiments will be apparent to those skilled in the art and may be made without departing from the scope or spirit of these disclosures. Although the subject matter has been described in terms of exemplary embodiments, it is not limited thereto. Rather, the appended claims should be construed broadly to include other variants and embodiments which can be made by those skilled in the art.

What is claimed is:

1. A system comprising:
a non-transitory computer readable medium storing instructions; and
one or more processors communicatively coupled to the non-transitory computer readable medium and configured to execute the instructions to:
execute an artificial intelligence agent based on a received signal identifying a domain-specific clinical context;
generate, using the executed artificial intelligence agent, inquiry data characterizing an inquiry associated with the domain-specific clinical context;
transmit the inquiry data to a user device;
receive response data from the user device;
generate, based on the inquiry data and the response data, annotated data in a structured format corresponding to the domain-specific clinical context;
generate a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities;
input the first dual prompt to a large language model and generate the data object;
generate a second dual prompt comprising a third prompt to generate a normalized clinical schema, and a fourth prompt to correct inconsistencies of the data object;
input the second dual prompt to the large language model and generate the normalized clinical schema;
generate graph node data based on the normalized clinical schema, wherein the graph node data includes nodes representing the identities and edges representing the relationships of the identities;
generate graphical user interface elements based on the graph node data; and
transmit the graphical user interface elements for display.

2. The system of claim 1 wherein the domain-specific clinical context identifies one of a cardiac context and a cancer context.

3. The system of claim 2 wherein the one or more processors are configured to execute the instructions to:
determine, based on the received signal, the one of the cardiac context and the cancer context; and
configure the artificial intelligence agent for execution based on the determined one of the cardiac context and the cancer context.

4. The system of claim 1 wherein the one or more processors are configured to execute the instructions to apply a text-to-speech process to the inquiry data to generate inquiry audio data, wherein the transmitted inquiry data is the inquiry audio data.

5. The system of claim 1 wherein the response data is received as an audio signal, and wherein the one or more processors are configured to execute the instructions to apply a speech-to-text process to the audio signal to generate the response data.

6. The system of claim 1 wherein the first prompt indicates that the large language model is a clinical genetics assistant trained to extract structured family history information from transcripts, and a request to identify the identities, the relationships of the identities, and the clinical features of the identities.

7. The system of claim 1 wherein the second prompt indicates to extract (1) a list of all of the identities, (2) a list linking each of the identities to the clinical features, and (3) a natural language summary of the relationships of the identities.

8. The system of claim 1 wherein the second prompt indicates that the data object is to be generated in .JSON format.

9. The system of claim 1 wherein the third prompt indicates that the large language model is a data-structuring engine that converts a loosely structured family history into the normalized clinical schema, and a command to ensure relationship consistency, generational order, and gender assignment.

10. The system of claim 1 wherein the one or more processors are configured to execute the instructions to:
generate a fifth prompt comprising a request to output a clinician-friendly summary note that summarizes pedigree and risk findings based on the provided graph node data;
input the fifth prompt and the graph node data to the large language model and generate clinical data; and
generate the graphical user interface elements based on the clinical data.

11. A computer-implemented method, comprising:
executing an artificial intelligence agent based on a received signal identifying a domain-specific clinical context;
generating, using the executed artificial intelligence agent, inquiry data characterizing an inquiry associated with the domain-specific clinical context;
transmitting the inquiry data to a user device;
receiving response data from the user device;
generating, based on the inquiry data and the response data, annotated data in a structured format corresponding to the domain-specific clinical context;
generating a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities;
inputting the first dual prompt to a large language model and generate the data object;
generating a second dual prompt comprising a third prompt to generate a normalized clinical schema, and a fourth prompt to correct inconsistencies of the data object;
inputting the second dual prompt to the large language model and generating the normalized clinical schema;
generating graph node data based on the normalized clinical schema, wherein the graph node data includes nodes representing the identities and edges representing the relationships of the identities;
generating graphical user interface elements based on the graph node data; and
transmitting the graphical user interface elements for display.

12. The computer-implemented method of claim 11 wherein the domain-specific clinical context identifies one of a cardiac context and a cancer context.

13. The computer-implemented method of claim 12 comprising:
determining, based on the received signal, the one of the cardiac context and the cancer context; and
configuring the artificial intelligence agent for execution based on the determined one of the cardiac context and the cancer context.

14. The computer-implemented method of claim 11 comprising applying a text-to-speech process to the inquiry data to generate inquiry audio data, wherein the transmitted inquiry data is the inquiry audio data.

15. The computer-implemented method of claim 11, wherein the response data is received as an audio signal, the method comprising applying a speech-to-text process to the audio signal to generate the response data.

16. The computer-implemented method of claim 11 wherein the first prompt indicates that the large language model is a clinical genetics assistant trained to extract structured family history information from transcripts, and a request to identify the identities, the relationships of the identities, and the clinical features of the identities.

17. The computer-implemented method of claim 11 wherein the second prompt indicates to extract (1) a list of all of the identities, (2) a list linking each of the identities to the clinical features, and (3) a natural language summary of the relationships of the identities.

18. The computer-implemented method of claim 11, wherein:
the second prompt indicates that the data object is to be generated in . JSON format; and
the third prompt indicates that the large language model is a data-structuring engine that converts a loosely structured family history into the normalized clinical schema, and a command to ensure relationship consistency, generational order, and gender assignment.

19. The computer-implemented method of claim 11, comprising:
generating a fifth prompt comprising a request to output a clinician-friendly summary note that summarizes pedigree and risk findings based on the provided graph node data;
inputting the fifth prompt and the graph node data to the large language model and generate clinical data; and
generating the graphical user interface elements based on the clinical data.

20. A non-transitory computer-readable medium comprising instructions that, when read by one or more processors, cause the one or more processors to:
execute an artificial intelligence agent based on a received signal identifying a domain-specific clinical context;
generate, using the executed artificial intelligence agent, inquiry data characterizing an inquiry associated with the domain-specific clinical context;
transmit the inquiry data to a user device;
receive response data from the user device;
generate, based on the inquiry data and the response data, annotated data in a structured format corresponding to the domain-specific clinical context;
generate a first dual prompt comprising a first prompt to identify identities, relationships of the identities, and clinical features of the identities, and a second prompt to generate a data object based on the annotated data, wherein the data object identifies the identities, the relationships of the identities, and the clinical features of the identities;
input the first dual prompt to a large language model and generate the data object;
generate a second dual prompt comprising a third prompt to generate a normalized clinical schema, and a fourth prompt to correct inconsistencies of the data object;
input the second dual prompt to the large language model and generate the normalized clinical schema;
generate graph node data based on the normalized clinical schema, wherein the graph node data includes nodes representing the identities and edges representing the relationships of the identities;
generate graphical user interface elements based on the graph node data; and
transmit the graphical user interface elements for display.
